(19) United States
(12) Patent Application Publication
(10) Pub. No.: US 2026/0018249 A1
(43) Pub. Date: Jan. 15, 2026

(54) PEPTIDE MANUFACTURABILITY DETERMINATION

(71) Applicant: AMAZON TECHNOLOGIES, INC., RENO, NV (US)

(72) Inventors: Anta IMATA SAFO, Seattle, WA (US); Samuel Anthony DANZIGER, Seattle, WA (US); Gil SADEH, Herzliya (IL); Layne Christopher PRICE, Seattle, WA (US); Frank Wilhelm SCHMITZ, Seattle, WA (US); David HECKERMAN, Bellevue, WA (US); Haibao TANG, Cupertino, CA (US); Alena HARLEY, Los Gatos, CA (US)

(21) Appl. No.: 18/725,548

(22) PCT Filed: May 30, 2024
(86) PCT No.: PCT/US2024/031773

§ 371 Date: Jun. 28, 2024

Related U.S. Application Data
(60) Provisional application No. 63/505,231, filed on May 31, 2023.

(51) Int. Cl. G16B 40/00

(52) U.S. Cl. CPC G16B 40/00

(57) ABSTRACT

Approaches for predicting manufacturability of a peptide are provided. A request for information related to manufacturability of a peptide can be received. A determination as to whether the peptide is predicted to be synthesizable can be made, such as by using a machine learning model. The machine learning model can be trained on data including manufacturer specifications and descriptions associated with a peptide and features for peptides. A second determination can be made as to whether the peptide is predicted to be soluble, using the same or different machine learning model trained with solubility data for peptides. If the peptide is predicted to be soluble and synthesizable, a manufacturability score for the peptide can be determined. The manufacturability score can correspond to or be indicative of a chance of successfully manufacturing the peptide.

### CROSS-REFERENCE TO RELATED APPLICATION

The present application claims the benefit of U.S. Provisional Patent Application No. 63/505,231, filed May 31, 2023, the entire contents of which are incorporated herein by reference in their entirety for all purposes.

### BACKGROUND

A peptide is a short chain of amino acids that can be used for various biological purposes. Peptides can be artificially manufactured for multiple applications, such as in drug development or use in supplements. Depending on the specifics of the manufacturing process, factors related to the peptide length and sequence may prevent a substantial percentage of peptides from being properly synthesized. Other factors such as pH, temperature, and storage conditions can all affect the stability of a peptide in solution. For a manufactured peptide to be usable, it must be correctly synthesized and solubilized. Therefore, to minimize manufacturing failures, there needs to be a way to accurately predict whether a peptide can be successfully manufactured.

### BRIEF DESCRIPTION OF THE DRAWINGS

Various embodiments in accordance with the present disclosure will be described with reference to the drawings, in which:

FIG. 1 illustrates an example method that can be used in accordance with the various embodiments.

FIG. 2 illustrates an example representation of a manufacturability analysis system that can be utilized in accordance with the various embodiments.

FIGS. 3A and 3B illustrate example histograms of predicted manufacturability in accordance with the various embodiments.

FIG. 4 illustrates an example receiver operating characteristic curve for predicted manufacturability in accordance with the various embodiments.

FIG. 5 illustrates an example calibration curve for converting a manufacturability score to probability of manufacturability in accordance with the various embodiments.

FIG. 6 illustrates an example active learning system that can be utilized to implement one or more aspects of the various embodiments.

FIG. 7 illustrates an example decision tree showing how factors derived from peptides can be used to determine a manufacturability score in accordance with the various embodiments.

FIG. 8 illustrates an example method that can be used in accordance with the various embodiments.

FIG. 9 illustrates components of an example computing device that can be utilized in accordance with various embodiments.

FIG. 10 illustrates an example of an environment for implementing aspects in accordance with various embodiments.

FIG. 11 illustrates components of another example environment in which aspects of various embodiments can be implemented.

FIGS. 12A and 12B are example histograms illustrating the expected number of peptides manufactures based on a manufacture simulation process, in accordance with various embodiments.

FIGS. 13A and 13B are example histograms illustrating a comparison between manufacturing simulation methods optimized for different criteria, in accordance with various embodiments.

### DETAILED DESCRIPTION

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

A manufacturer may provide a list of heuristics or criteria that can be used to determine whether a peptide would be manufacturable. Manufacturability, as used herein in the context of peptide synthesis, may refer to the process that predicts whether a peptide can be practically and reliably manufactured for research, therapeutic, or commercial purposes. In one embodiment, manufacturability may be determined by a process that assesses and/or predicts one or more features associated with a peptide, wherein the features may include one or more of synthesizability, solubility, quality prediction, and other factors that may affect the production of the peptide. For example, the manufacturer may indicate that the peptide should not have too many hydrophobic amino acids. However, if there is an amino acid that increases hydrophobicity but also increases rigidity (because rigid peptides may be more likely to be soluble in certain solvents), a decision can be made as to whether the tradeoff is worth using the amino acid for manufacturing. For example, in aqueous (water-based) solutions, increased hydrophobicity typically reduces solubility, whereas increased rigidity may counteract this effect to some extent by stabilizing the peptide structure. In other solvents, such as nitrogen-based solvents (e.g., nitromethane) or organic solvents (e.g., ethanol), the effects of hydrophobicity and rigidity may differ. Rigidity may enhance or diminish solubility depending on the specific chemical interactions between the peptide and the solvent. Therefore, the amino acids selected for peptide synthesis may require consideration of the intended solvent environment, which may include assessment of both hydrophobicity and rigidity in relation to solubility and overall manufacturability in various solvent systems. A major factor contributing to rigidity is the existence of the amino acid proline, which has a five-atom ring that essentially makes it much less bendable than other amino acids. It is hypothesized that proline (and other amino acids that make the peptide more rigid) keep the peptide in solution (i.e., soluble) because rigid peptides cannot easily bend to form bonds with other peptides. When peptides aggregate, peptides tend to fall out of solution and fail manufacturability, and cannot be used in a final product. Further, a solution containing a given peptide may change depending on the use case, so solubility may also change in response depending on the use case. Additionally, given the vast number of potential peptide sequences, each having their own individual features for analysis, it would be difficult, if not impossible, to determine with accuracy what tradeoffs to make without use of a computer-based algorithm. For example, there can be over thousands of sequences for synthesis, each having hundreds of features for analysis, which would be too much data for a human to predict with the level of accuracy required for manufacturing. Additionally, in using a computer-based algorithm, manufacturability can be assessed in a fully objective manner leaving less room for error. Additionally, a manufacturer may be tasked with creating a mixture of peptides in a specific solution. In one embodiment, individual peptides may be independently soluble, but they may aggregate when combined together in solution and become unusable. Computer-based algorithms (e.g., machine learning algorithms) can be used to select peptides that are co-soluble.

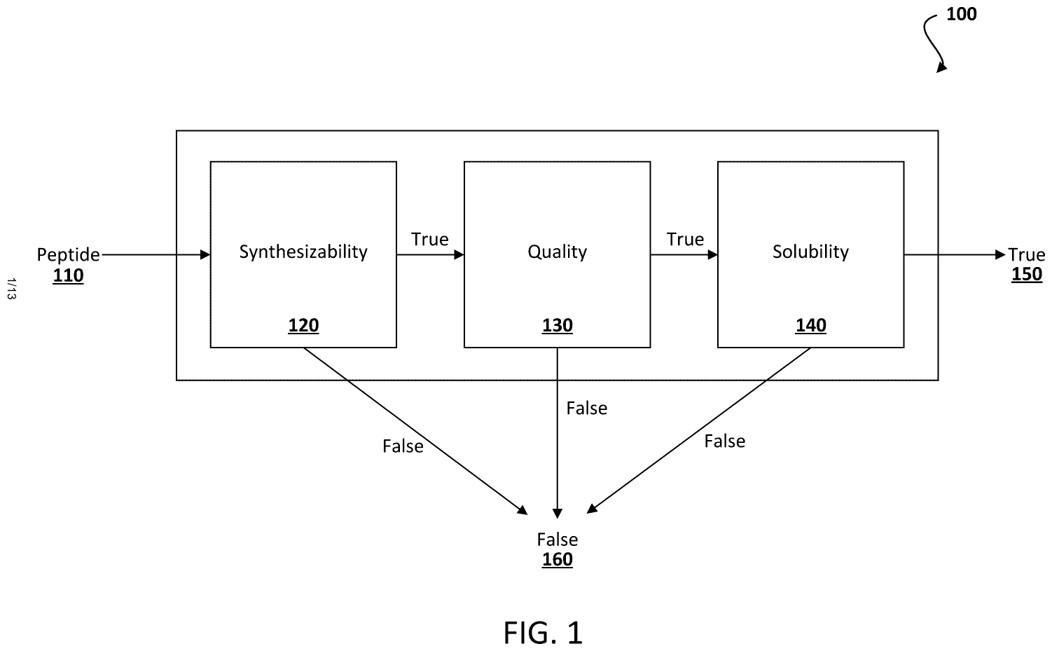

FIG. 1 illustrates an example method 100 that can be used in accordance with the various embodiments. In accordance with the various embodiments, a request for information related to manufacturability of a peptide can be received. A determination as to whether the peptide is predicted to be synthesizable can be made, such as by using a machine learning model. The machine learning model can be trained on data including one or more of manufacturer specifications, descriptions associated with a peptide, and features of the peptides. A second determination can be made as to whether the peptide is predicted to be soluble, using the same or different machine learning model trained with solubility data for peptides. If the peptide is predicted to be soluble and synthesizable, a manufacturability score for the peptide can be determined. The manufacturability score can correspond to or be indicative of a chance of manufacturability of the peptides. In some exemplary embodiments, a determination can also be made as to whether the peptide will likely pass a quality check, which traditionally is determined using mass spectrometry techniques. The quality check ensures that the peptide produced actually is the peptide that was initially requested. The individual predictions for whether the peptide is synthesizable, soluble, and passes a quality check can be made using the same or a different machine learning model. Although FIG. 1 illustrates an exemplary order for different phases of predictions and assessment, it should be understood that the sequence in which these evaluations are conducted can vary, or may happen simultaneously. The order of operations, whether predicting synthesizability, solubility, quality, or other features, may be vary depending on specific manufacturing contexts or the availability of data.

In the example illustrated in FIG. 1, a request for generation of a peptide 110 or peptide representation may be input to a model, such as a hierarchical model, to assess manufacturability by a manufacturer. The synthesizability 120 of the peptide can be assessed. If the peptide is predicted to be synthesizable, it can then be determined whether the peptide passes quality control checks 130. If it is determined that the peptide is not predicted to be synthesizable, then the analysis for manufacturability fails with a false response 160. As used herein, synthesizability may refer to the feasibility and efficiency with which a peptide can be chemically synthesized based on its amino acid sequence and structural characteristics. In one embodiment, synthesizability may depend on the practicality of chemically producing a peptide based on its amino acid sequence and structural properties. For example, factors affecting synthesizability may include the specific amino acids involved, which can influence the stability and reactivity of the peptide during synthesis. In one embodiment, synthesizability may also depend on the peptide's thermal stability, its reaction to pH variations, or its interaction with other molecular components in a formulation. In another example, sequences prone to forming complex secondary structures or containing chemically sensitive residues such as cysteine or asparagine may complicate synthesis and purification processes. Additionally, the length of the peptide chain may also be a factor, as longer sequences may face higher rates of synthesis errors and challenges in purification.

In the quality control phase, the process may involve using a predictive model to estimate whether the synthesized peptide will meet the specified quality criteria. This model-based prediction may serve as an initial check before actual manufacturing, to assess the likelihood of the peptide passing quality control. In some embodiments, after the peptide is manufactured, its quality can be validated using mass spectrometry techniques. Such an empirical validation may not only confirm the model's predictions but also provide essential data that can be used to refine the predictive model further. If the model predicts that a peptide will fail the quality check, and this is confirmed through mass spectrometry, the manufacturability assessment of the peptide is deemed unsuccessful, resulting in a false response 160 in the model's output. In one embodiment, a determination can be informed by data generated through mass spectrometry techniques. Mass spectrometry can be used to analyze the composition and structure of synthesized peptides, providing high-quality data that is used to train the predictive model. By incorporating mass spectrometry data, the model learns to accurately predict the quality of peptides, ensuring that the peptide produced matches the peptide that was initially specified in the design.

If the synthesized peptide passes quality control, it can then be determined whether the synthesized peptide will be soluble 140 in a solution. In one embodiment, solubility may be influenced by the interaction between the peptide's chemical characteristics and the solvent used. The choice of solvent may significantly affect solubility, as peptides composed of hydrophilic amino acids, such as lysine and arginine, are typically more soluble in polar solvents such as water, while peptides with hydrophobic amino acids, such as leucine and valine, may dissolve better in non-polar solvents. For example, the solvent's polarity should be compatible with the peptide's polarity to enhance dissolution. In one embodiment, solubility may also be affected by a peptide's length, with shorter chains tending to be more soluble, and the ionic strength and pH of the solution, which can alter solubility by influencing the peptide's charge. If the synthesized peptide is predicted to be soluble, then the requested peptide 110 can be manufactured with a true value 150. If the synthesized peptide is not predicted to be soluble, then the analysis for manufacturability fails with a false response 160. False responses can be added to a training data set for the model to augment the data set and improve peptide manufacturability predictions for future requests through active learning techniques.

In one embodiment, such a hierarchical model is especially beneficial in cases where a data set may only have a small number of samples. If a sample is determined to be insoluble, non-synthesizable, or fails to pass a quality check, that data would typically be excluded and not used in any meaningful way. Through use of the hierarchical model, all of the data can be used to inform a better decision by the model as a whole. Individual decisions for synthesizability 120, quality 130, and solubility 140 may all be their own intermediary model that informs a larger machine learning model as to manufacturability of a requested peptide 110. For example, positive data corresponding to decisions resulting in favorable outcomes can also be added to the training data set. Solubility and synthesizability data is often not known for a given peptide, so the active learning process for the hierarchical model can improve manufacturability predictions over time.

A binary prediction task of predicting manufacturability can be broken down to more granular decisions in this way to infer relevant labels based on the task hierarchy. For example, if a peptide was manufactured successfully then a positive label can be associated for all sub-tasks (e.g., synthesizability, solubility, and quality check). In some cases all labels might not be known. For example, if a certain peptide failed at an early stage the manufacturer might not have tested it at later stages.

By using the hierarchical model described herein, different sub-tasks can be trained separately (e.g., using a different model for each sub-task trained individually) or jointly (e.g., using a single model trained with multi-tasks on all sub-tasks). Training the three sub-tasks and one parent task can be done with four binary classifiers, with one binary classifier for each task. The sub-tasks can correspond to prediction of synthesizability, quality check, and prediction of solubility of a given peptide, and the parent task can correspond to prediction of manufacturability for the peptide. Alternatively, a multi-class classifier can be used to cover all hierarchy options, with classes indicating whether the peptide failed during synthesis prediction, whether the peptide passed during synthesis prediction but failed quality check, whether the peptide passed synthesis prediction and quality check but failed at solubility prediction, and whether the peptide passed all stages. The different stages can be tested in any order depending on specific context and/or different factors.

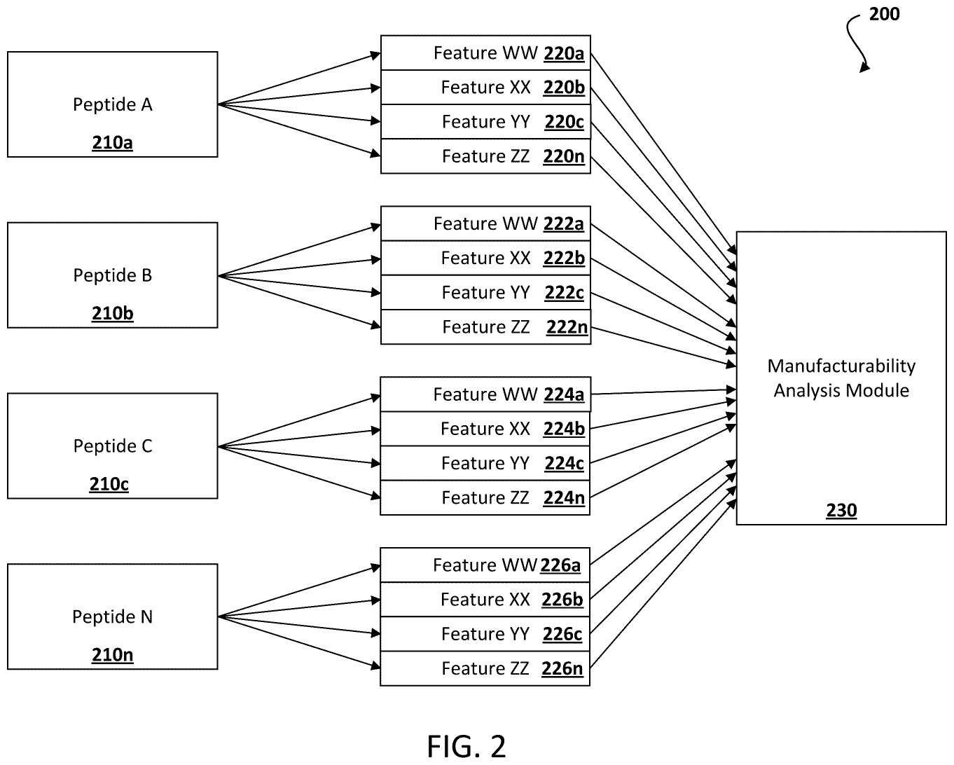

FIG. 2 illustrates an example representation 200 of a manufacturability analysis system that can be utilized in accordance with the various embodiments. In this example, a set of peptide samples 210a, 210b, 210c, 210n can be requested for analysis and manufacture. While this example only shows four peptide samples, any “N” number of samples can be requested, such as thousands of peptide samples. Each peptide sample can have hundreds of features (e.g., “220a-n, 222a-n, 224a-n, 226a-n” of FIG. 2) that are provided for consideration to a manufacturability analysis module 230. Features may include, but are not limited to, amino acid sequencing information, length, thermostability, molecular weight, charge, polarity, hydrophobicity, hydrogen bonding, counts of specific amino acids, isoelectric properties, topological descriptors, secondary structure, rigidity, conformation, post-translational modifications, other heuristics that influence manufacturability, and meta-features derived from principal component (and related) dimension reduction strategies. Such features may be used to describe the overall peptide composition, the amino acids on the N and C terminus of the peptide, and the minimum/maximum of a fixed sliding window run over the peptide. Overall, there are hundreds or more features and descriptors that can be analyzed for each peptide.

The manufacturability analysis module 230 may analyze peptides and their associated features to assess manufacturability. The manufacturability analysis module 230 may assess features such as synthesizability, quality control, and solubility in the manner explained herein with respect to FIG. 1. Given the complex data analysis involved with the high number of input peptides and high number of features for each peptide, the manufacturability analysis module 230 may suggest decisions regarding peptide manufacturability in an efficient and objective manner, improving accuracy and enabling intelligent decision-making for peptide manufacturing. For example, if an amino acid is added to a peptide that increases hydrophobicity but also increases rigidity, the model underlying the manufacturability analysis module 230 may determine with precision just how much hydrophobicity and rigidity are increased, to inform a better decision about whether the peptide is worth manufacturing. A lab technician would likely not be able to determine such information with as much precision and accuracy as the model.

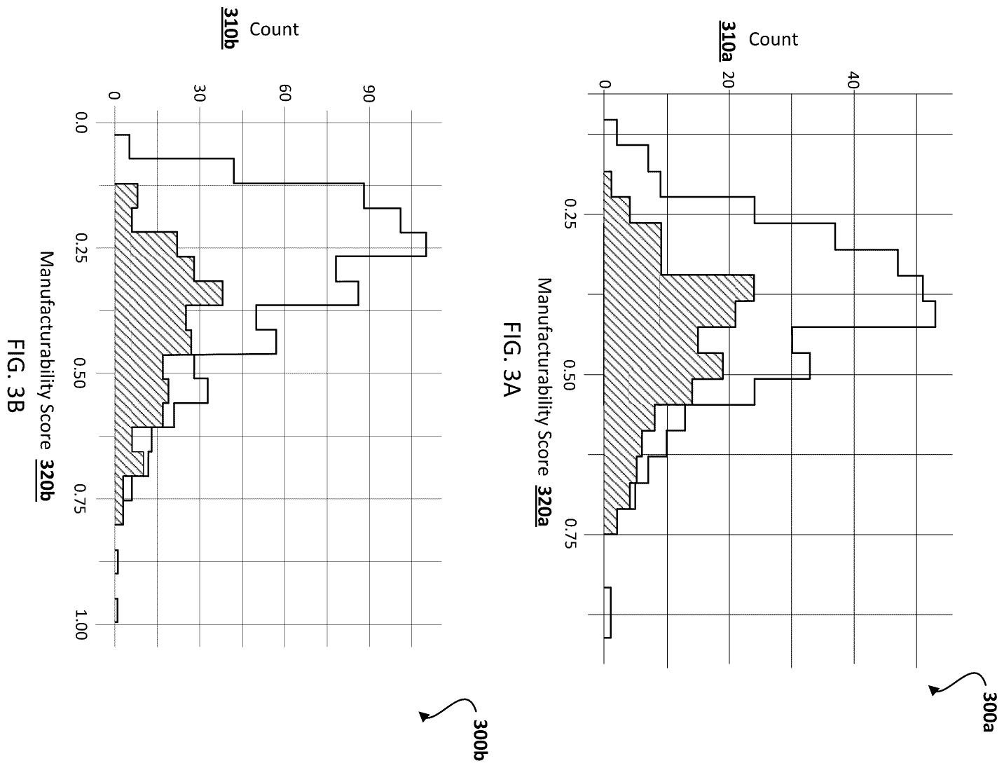

FIGS. 3A and 3B illustrate example histograms 300a, 300b for predicting manufacturability in accordance with the various embodiments. FIGS. 3A and 3B illustrate the manufacturability predictions for peptides, visualized through two different versions of a predictive model.

In FIG. 3A, the horizontal axis 320a represents the predicted manufacturability scores, ranging from 0.0 to 1.0, where scores closer to 1.0 suggest a higher likelihood of successful manufacturability. The vertical axis 310a shows the count of peptides, indicating how many peptides are predicted to fall within each score bracket. The empty outline bars on the histogram represent the total number of peptides subjected to the manufacturing process based on these predictions, while the shaded areas within these bars show how many of these peptides were actually successfully manufactured. FIG. 3A provides a visual representation of prediction success against actual outcomes. The alignment or overlap between the empty bars and the shaded areas within these bars illustrates effectiveness of the model. Where the shaded area covers most of the empty bar, most of the peptides in that score bin were successfully manufactured. For a good manufacturability predictor, as the Manufacturability Score increases the fraction of the empty bar covered by the shaded area will increase. In one embodiment, such a model may utilize the “Leave One Out” (LOO) cross-validation process to ensure accuracy, especially in scenarios with limited data. LOO process may most accurately reveal the model's quality by using each data point in the dataset one at a time as the test set while the rest of the data serves as the training set. This method is beneficial in scenarios with sparse data because it maximizes the use of available data for training and testing, providing a thorough assessment of the model's predictive capabilities.

FIG. 3B illustrates another histogram corresponding to an updated model that includes more descriptors for enhanced accuracy. The horizontal axis 320b may show the manufacturability scores from 0.0 to 1.0, and the vertical axis 310b the peptides count per predicted score. The empty outline bars illustrate the total peptides processed, and the shaded areas represent those that were successfully manufactured. In one embodiment, the example histogram 300b accounts for additional manufacturability data and uses a “Least Absolute Shrinkage and Selection Operator” (LASSO) regression model and a strict LOO cross validation. In one embodiment, LASSO regression is a type of linear regression that performs both variable selection and regularization in order to enhance the prediction accuracy and interpretability of the statistical model it produces. In one embodiment, the underlying model illustrated in FIG. 3B may include more descriptors than the model corresponding to FIG. 3A. When dealing with data sets involving a large number of predictors (variables), LASSO regression may be used to impose a penalty on the absolute size of the regression coefficients. The underlying model may account for features such as a number of specific types of amino acids on the c- or n-terminus of the peptide.

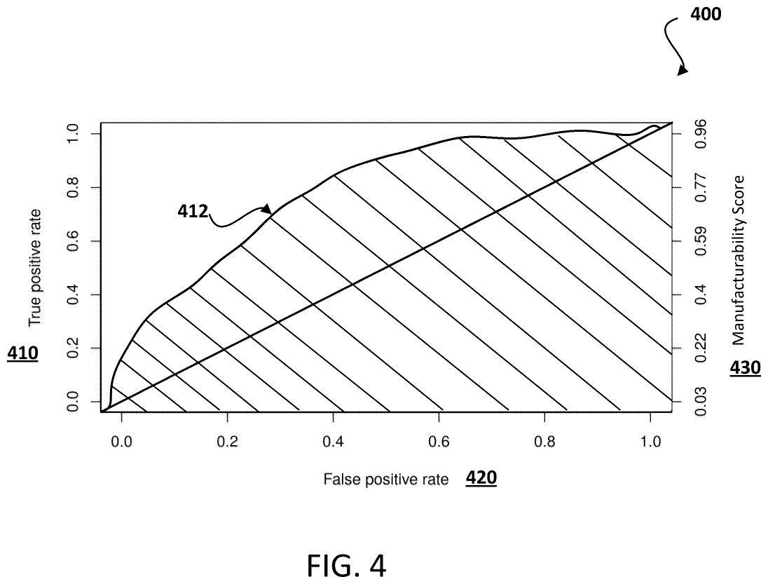

FIG. 4 illustrates an example receiver operating characteristic (ROC) curve 412 for predicting solubility in accordance with the various embodiments. In one embodiment, the ROC curve 412 is used to evaluate the performance of a predictive model in terms of its ability to classify outcomes correctly. The curve plots the true positive rate (TPR) on the vertical axis 410 against the false positive rate (FPR) on the horizontal axis 420, which demonstrates the trade-offs between benefits (true positives) and costs (false positives). The TPR, also known as sensitivity, indicates the model's ability to correctly identify actual positives. The FPR, in contrast, measures the proportion of negatives that are incorrectly identified as positives. The ROC curve 412 of this example uses the same data as in the histograms of FIGS. 3A and 3B. When moving across various manufacturability scores 430 at a particular point, the true positive rate 410 and the false positive rate 420 can be analyzed against the manufacturability scores 430. A model's effectiveness is generally gauged by an area under the ROC curve (AUC). For example, a perfect model would score an AUC of 1, indicating it perfectly distinguishes between manufacturable and non-manufacturable peptides. In practical terms, analyzing this curve enables the selection of an appropriate threshold score that maximizes the model's accuracy, which ensures that the manufacturing process focuses resources on peptides most likely to be successfully synthesized while avoiding expenditure on those that are not. In FIG. 4, the manufacturability score of the ROC curve 412 has a shaded area under the curve (AUC) value of ˜0.80. The true positive rate 410 is indicative of a measure of the proportion of actual positive cases that were correctly manufactured. The false positive rate 420 is a measure of the proportion of actual negative cases that were not manufactured.

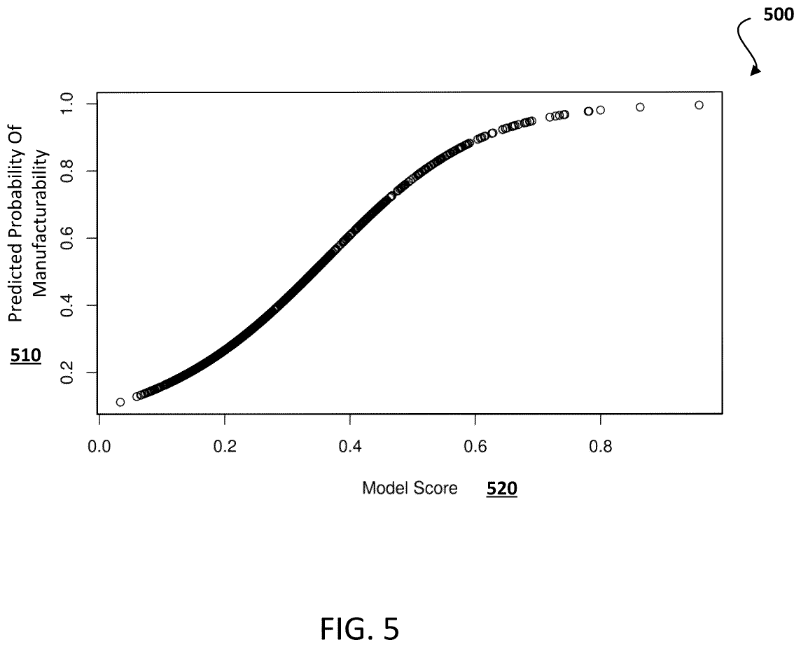

FIG. 5 presents a calibration curve that translates model scores into predicted probabilities of peptide manufacturability. This curve may interpret model output, which may provide scores not directly expressed as percentages. The horizontal axis represents Model Score 520 and displays the scores generated by the model, which could range from 0 to 1 in this example. In one embodiment, the model score may be a number in other ranges, such as from 0 to 12 in other model variants. For such models, conversion from model scores to predicted probability is necessary (such as transforming a model score of 11.4 into a probability. The vertical axis represents predicted probability of manufacturability 510 and indicates the predicted likelihood that a peptide can be successfully manufactured based on the model's output.

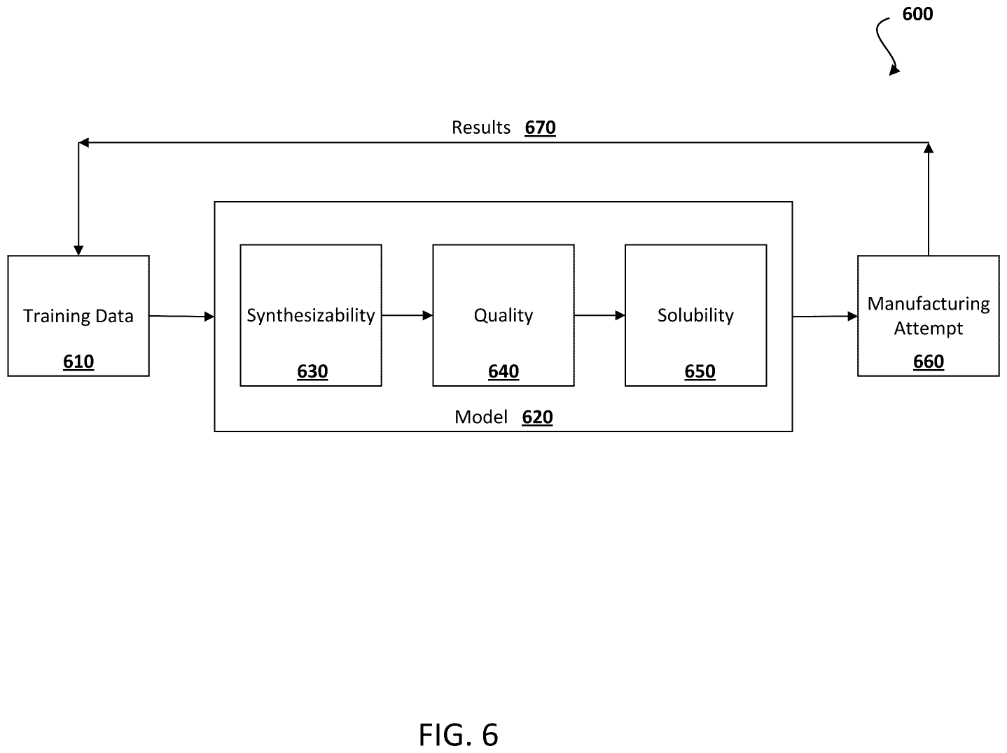

FIG. 6 illustrates an example active learning system 600 that can be utilized to implement one or more aspects of the various embodiments. Active learning, as it applies to the various embodiments described herein, may be referred to as the process underlying a model that chooses a peptide for manufacture that will most improve the model. For example, a peptide X may have an 80% manufacturability score, whereas peptide Y may have a 60% manufacturability score. Independent of these scores, peptide X may have a “curiosity” score of 1 which means that the model is certain of peptide X's 80% manufacturability score. In contrast, peptide Y may have a “curiosity” score of 9, which would indicate a high uncertainty that peptide Y has a 60% manufacturability score. For peptides of high uncertainty, like with peptide Y in the example above, peptide Y can be manufactured and the results of manufacturability can be fed into a model, such as a machine learning model. Further, in accordance with one or more embodiments described herein, specific peptides may be requested for manufacture specifically to improve the set of training data.

In accordance with this example, a set of training data 610 can be associated with a model 620. The model 620 may be a machine learning model trained to predict or otherwise assess manufacturability of a peptide. The training data 610 may include, but is not limited to, peptides and peptide representations, peptide feature or heuristics/criteria, manufacturer specifications and descriptors, and solubility. The training data can be correlated using a single value indicative of a particular aspect of the training data. For example, a heuristic or criterion can be correlated using a value or series of values indicative of the heuristic or criterion. The model 620 may include, but is not limited to, a synthesizability analysis module 630, a quality control analysis module 640, and a solubility analysis module 650. The synthesizability analysis module 630 can be utilized, in accordance with one or more embodiments, to assess whether the peptide in the input data is predicted to be synthesizable. If the peptide is predicted to be synthesizable, it can then be determined whether the peptide likely will pass quality control checks using quality control analysis module 640. For quality control, the model can determine whether the synthesizable peptide likely matches the requested peptide. In one embodiment, the model may use a machine learning model for predicting whether the peptide likely matches the requested peptide. After the peptide is synthesized, in some exemplary embodiments, the quality of the synthesized peptide can be checked using mass spectrometry techniques, where the mass spectrometry is used as the training data in training the model that predicts synthesizability. If the synthesizable peptide is predicted to pass quality control, it can then be determined whether the synthesizable peptide is predicted to be soluble in a solution using solubility analysis module 650. If the synthesizable peptide is predicted to be soluble, then the peptide 660 can be attempted to be manufactured. The individual predictions for whether the peptide is synthesizable, soluble, and passes a quality check can be made using the same or a different machine learning model. The order of the individual predictions can vary based on specific context. Further, positive data corresponding to decisions resulting in favorable outcomes can also be added to a training data set associated with the machine learning model(s).

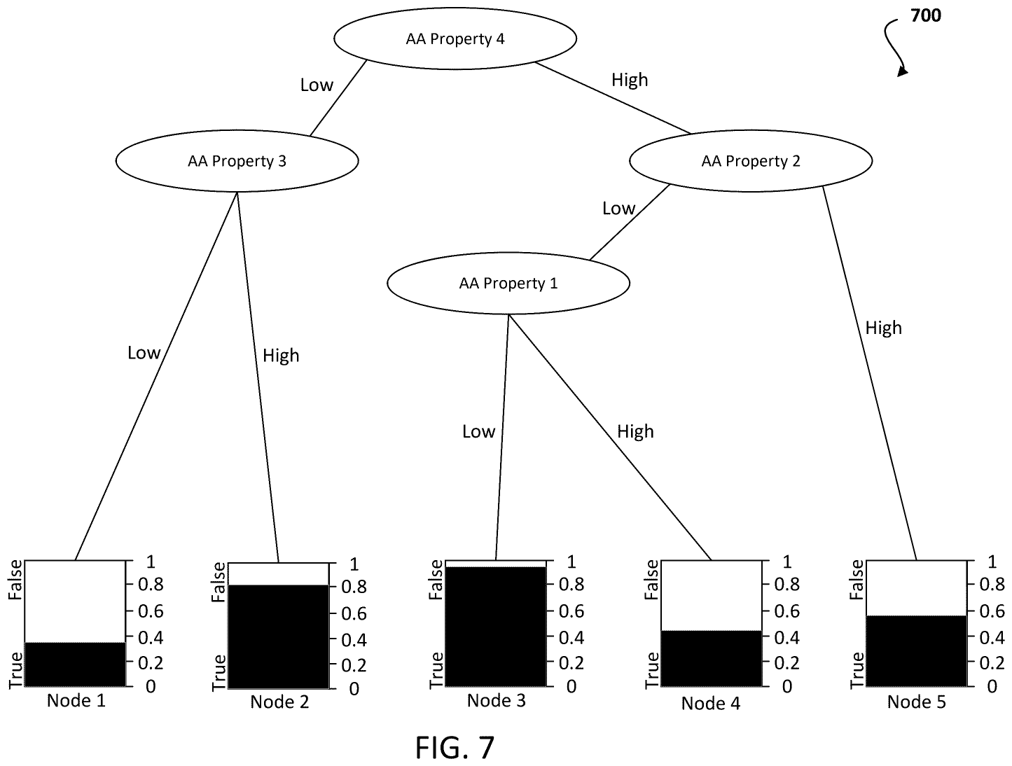

In some cases, it can be determined that a peptide is not predicted to be synthesizable, fails the predicted quality check, is predicted to not be soluble, or otherwise fails at manufacturability. These results 670 can be added to the training data set to augment the data set and improve peptide synthesizability predictions for future requests through active learning techniques. Such a hierarchical model is especially beneficial in cases where a data set may only have a small number of samples. If a sample is determined to not be soluble, that data would typically get excluded and not used in any meaningful way. Through use of the hierarchical model, all of the data can be used to inform a better decision by the model as a whole. The synthesizability analysis module 630, quality control analysis module 640, and solubility analysis module 650 may all have their own intermediary model that informs a larger machine learning model as to manufacturability of a requested peptide. Solubility data is often not known for a given peptide, so the active learning process for the hierarchical system can improve manufacturability predictions over time. FIG. 7 illustrates an example decision tree 700 that can be utilized in accordance with the various embodiments. A decision tree, such as decision tree 700 of FIG. 7, can provide additional insights as to why a peptide is or is not manufacturable. Various amino acid (AA) properties or features (“AA Property 1, AA Property 2, AA Property 3, AA Property 4) can be analyzed against various thresholds (“low” and “high”). In accordance with an exemplary embodiment, an AA property can include, but is not limited to, hydrophobicity, charge, side chain properties, acid-base properties, stereochemistry, hydrogen bonding, and chemical reactivity. Based on the data provided in the decision tree 700, it can be determined how likely it is that a soluble peptide can be successfully created. The decision tree 700 can provide at various nodes (“Node 1, Node 2, Node 3, Node 4, Node 5”) information about how likely the peptide is to be successfully manufactured. For example, if AA Property 4 is low but AA Property 3 is high, there is a higher chance at successfully manufacturing a peptide than if AA Property 3 were also low. If AA Property 4 was high, AA Property 2 was low, and AA Property 1 was also low, then there is a very good chance of successfully manufacturing the peptide. The decision tree can provide insights as to which specific properties can cause a peptide to be successfully manufactured, which is not otherwise easily determinable by a technician given the vast number of features and amino acid sequences to analyze.

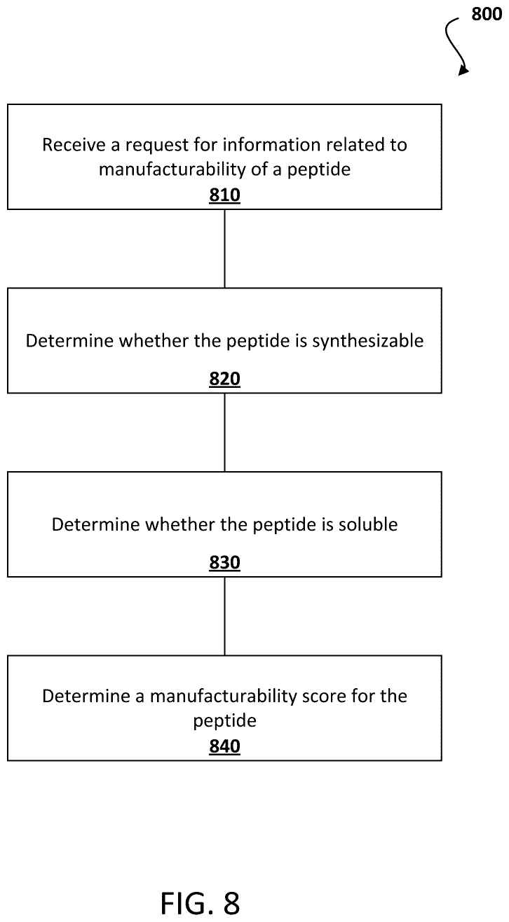

FIG. 8 illustrates an example method 800 that can be used in accordance with the various embodiments. It should be understood that for any process herein there can be additional, fewer, or alternative steps performed in similar or alternative orders, or in parallel, within the scope of the various embodiments unless otherwise specifically stated. A request for information related to manufacturability of a peptide can be received 810. A determination as to whether the peptide is synthesizable can be made 820, such as by using a machine learning model. The machine learning model can be trained on data including manufacturer specifications and descriptions associated with a peptide and features for peptides. A second determination can be made as to whether the peptide is soluble 830, using the same or different machine learning model trained with solubility data for peptides. If the peptide is soluble and synthesizable, a manufacturability score for the peptide can be determined 840. The manufacturability score can correspond to or be indicative of a chance of successfully manufacturing the peptide. In some exemplary embodiments, a determination can also be made as to whether the peptide is predicted to pass a quality check, which can be determined using mass spectrometry techniques. For example, a determination can be informed by data generated through mass spectrometry techniques. This process involves using mass spectrometry to analyze the composition and structure of synthesized peptides, providing high-quality data that is used to train the predictive model. By incorporating mass spectrometry data, the model learns to accurately predict the quality of peptides, ensuring that the peptide produced matches the peptide that was initially specified in the design. The quality check can help ensure that the peptide produced is actually the peptide that was initially requested.

In accordance with some exemplary embodiments, instead of explicitly extracting biochemical features for the peptides, deep learning can be used to predict biochemical features for the peptides through the use of embeddings for pretrained peptide/protein “language models,” protein structure prediction models, or a combination thereof. The deep learning model can have intermediate layers for each embedding of the peptide, and transferred learning can be used to take the embedded model and transfer it to perform a new task of making predictions using the embedded model.

Pretrained protein language models such as evolutionary scale modeling (ESM) can be used, in accordance with one or more embodiments, to extract informative peptide embeddings used to train a manufacturability prediction classifier. Alternatively, embeddings based on protein models after finetuning on large peptide datasets or embeddings based on structure prediction models can be used. An embedding derived from the pre-trained models can be complemented by known biochemical or physiochemical properties derived directly from the amino acid sequence and used as an input peptide representation for the manufacturability classifier.

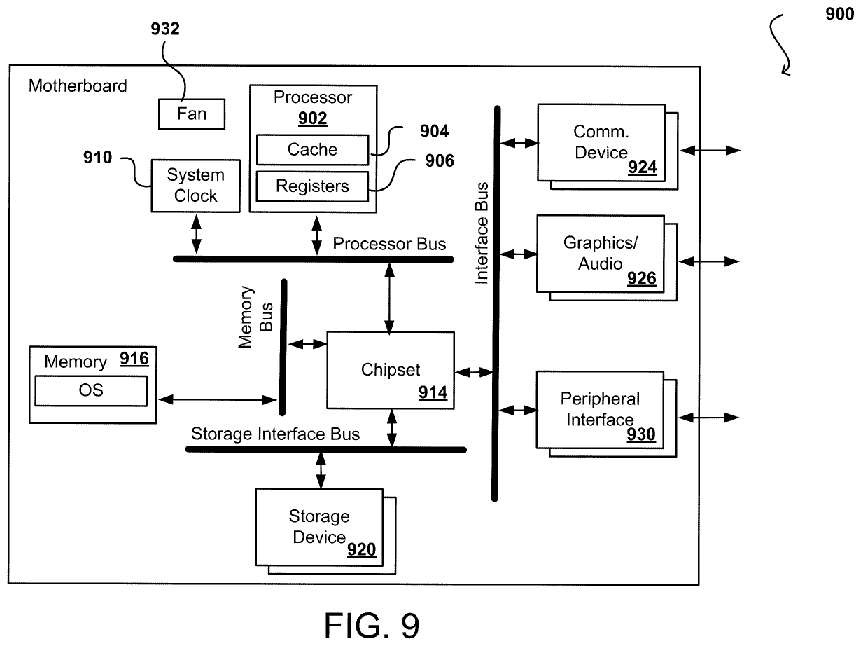

Computing resources, such as servers, that can have software and/or firmware updated in such a matter will generally include at least a set of standard components configured for general purpose operation, although various proprietary components and configurations can be used as well within the scope of the various embodiments. FIG. 9 illustrates components of an example computing device 900 that can be utilized in accordance with various embodiments. As known for computing devices, the computer will have one or more processors 902, such as central processing units (CPUs), graphics processing units (GPUs), and the like, that are electronically and/or communicatively coupled with various components using various buses, traces, and other such mechanisms. A processor 902 can include memory registers 906 and cache memory 904 for holding instructions, data, and the like. In this example, a chipset 914, which can include a northbridge and southbridge in some embodiments, can work with the various system buses to connect the processor 902 to components such as system memory 916, in the form or physical RAM or ROM, which can include the code for the operating system as well as various other instructions and data utilized for operation of the computing device. The computing device can also contain, or communicate with, one or more storage devices 920, such as hard drives, flash drives, optical storage, and the like, for persisting data and instructions similar, or in addition to, those stored in the processor and memory. The processor 902 can also communicate with various other components via the chipset 914 and an interface bus (or graphics bus, etc.), where those components can include communications devices 924 such as cellular modems or network cards, media components 926, such as graphics cards and audio components, and peripheral interfaces 930 for connecting peripheral devices, such as printers, keyboards, and the like. At least one cooling fan 932 or other such temperature regulating or reduction component can also be included as well, which can be driven by the processor or triggered by various other sensors or components on, or remote from, the device. Various other or alternative components and configurations can be utilized as well as known in the art for computing devices.

At least one processor 902 can obtain data from physical memory 916, such as a dynamic random access memory (DRAM) module, via a coherency fabric in some embodiments. It should be understood that various architectures can be utilized for such a computing device, that may include varying selections, numbers, and arguments of buses and bridges within the scope of the various embodiments. The data in memory may be managed and accessed by a memory controller, such as a DDR controller, through the coherency fabric. The data may be temporarily stored in a processor cache 904 in at least some embodiments. The computing device 900 can also support multiple I/O devices using a set of I/O controllers connected via an I/O bus. There may be I/O controllers to support respective types of I/O devices, such as a universal serial bus (USB) device, data storage (e.g., flash or disk storage), a network card, a peripheral component interconnect express (PCIe) card or interface 930, a communication device 924, a graphics or audio card 926, and a direct memory access (DMA) card, among other such options. In some embodiments, components such as the processor, controllers, and caches can be configured on a single card, board, or chip (i.e., a system-on-chip implementation), while in other embodiments at least some of the components may be located in different locations, etc.

An operating system (OS) running on the processor 902 can help to manage the various devices that may be utilized to provide input to be processed. This can include, for example, utilizing relevant device drivers to enable interaction with various I/O devices, where those devices may relate to data storage, device communications, user interfaces, and the like. The various I/O devices will typically connect via various device ports and communicate with the processor and other device components over one or more buses. There can be specific types of buses that provide for communications according to specific protocols, as may include peripheral component interconnect) PCI or small computer system interface (SCSI) communications, among other such options. Communications can occur using registers associated with the respective ports, including registers such as data-in and data-out registers. Communications can also occur using memory-mapped I/O, where a portion of the address space of a processor is mapped to a specific device, and data is written directly to, and from, that portion of the address space.

Such a device may be used, for example, as a server in a server farm or data warehouse. Server computers often have a need to perform tasks outside the environment of the CPU and main memory (i.e., RAM). For example, the server may need to communicate with external entities (e.g., other servers) or process data using an external processor (e.g., a General Purpose Graphical Processing Unit (GPGPU)). In such cases, the CPU may interface with one or more I/O devices. In some cases, these I/O devices may be special-purpose hardware designed to perform a specific role. For example, an Ethernet network interface controller (NIC) may be implemented as an application specific integrated circuit (ASIC) comprising digital logic operable to send and receive packets.

In an illustrative embodiment, a host computing device is associated with various hardware components, software components and respective configurations that facilitate the execution of I/O requests. One such component is an I/O adapter that inputs and/or outputs data along a communication channel. In one aspect, the I/O adapter device can communicate as a standard bridge component for facilitating access between various physical and emulated components and a communication channel. In another aspect, the I/O adapter device can include embedded microprocessors to allow the I/O adapter device to execute computer executable instructions related to the implementation of management functions or the management of one or more such management functions, or to execute other computer executable instructions related to the implementation of the I/O adapter device. In some embodiments, the I/O adapter device may be implemented using multiple discrete hardware elements, such as multiple cards or other devices. A management controller can be configured in such a way to be electrically isolated from any other component in the host device other than the I/O adapter device. In some embodiments, the I/O adapter device is attached externally to the host device. In some embodiments, the I/O adapter device is internally integrated into the host device. Also in communication with the I/O adapter device may be an external communication port component for establishing communication channels between the host device and one or more network-based services or other network-attached or direct-attached computing devices. Illustratively, the external communication port component can correspond to a network switch, sometimes known as a Top of Rack (“TOR”) switch. The I/O adapter device can utilize the external communication port component to maintain communication channels between one or more services and the host device, such as health check services, financial services, and the like.

The I/O adapter device can also be in communication with a Basic Input/Output System (BIOS) component. The BIOS component can include non-transitory executable code, often referred to as firmware, which can be executed by one or more processors and used to cause components of the host device to initialize and identify system devices such as the video display card, keyboard and mouse, hard disk drive, optical disc drive and other hardware. The BIOS component can also include or locate boot loader software that will be utilized to boot the host device. For example, in one embodiment, the BIOS component can include executable code that, when executed by a processor, causes the host device to attempt to locate Preboot Execution Environment (PXE) boot software. Additionally, the BIOS component can include or takes the benefit of a hardware latch that is electrically controlled by the I/O adapter device. The hardware latch can restrict access to one or more aspects of the BIOS component, such controlling modifications or configurations of the executable code maintained in the BIOS component. The BIOS component can be connected to (or in communication with) a number of additional computing device resources components, such as processors, memory, and the like. In one embodiment, such computing device resource components may be physical computing device resources in communication with other components via the communication channel. The communication channel can correspond to one or more communication buses, such as a shared bus (e.g., a processor bus, a memory bus), a point-to-point bus such as a PCI or PCI Express bus, etc., in which the components of the bare metal host device communicate. Other types of communication channels, communication media, communication buses or communication protocols (e.g., the Ethernet communication protocol) may also be utilized. Additionally, in other embodiments, one or more of the computing device resource components may be virtualized hardware components emulated by the host device. In such embodiments, the I/O adapter device can implement a management process in which a host device is configured with physical or emulated hardware components based on a variety of criteria. The computing device resource components may be in communication with the I/O adapter device via the communication channel. In addition, a communication channel may connect a PCI Express device to a CPU via a northbridge or host bridge, among other such options.

In communication with the I/O adapter device via the communication channel may be one or more controller components for managing hard drives or other forms of memory. An example of a controller component can be a SATA hard drive controller. Similar to the BIOS component, the controller components can include or take the benefit of a hardware latch that is electrically controlled by the I/O adapter device. The hardware latch can restrict access to one or more aspects of the controller component. Illustratively, the hardware latches may be controlled together or independently. For example, the I/O adapter device may selectively close a hardware latch for one or more components based on a trust level associated with a particular user. In another example, the I/O adapter device may selectively close a hardware latch for one or more components based on a trust level associated with an author or distributor of the executable code to be executed by the I/O adapter device. In a further example, the I/O adapter device may selectively close a hardware latch for one or more components based on a trust level associated with the component itself. The host device can also include additional components that are in communication with one or more of the illustrative components associated with the host device. Such components can include devices, such as one or more controllers in combination with one or more peripheral devices, such as hard disks or other storage devices. Additionally, the additional components of the host device can include another set of peripheral devices, such as Graphics Processing Units (“GPUs”). The peripheral devices and can also be associated with hardware latches for restricting access to one or more aspects of the component. As mentioned above, in one embodiment, the hardware latches may be controlled together or independently.

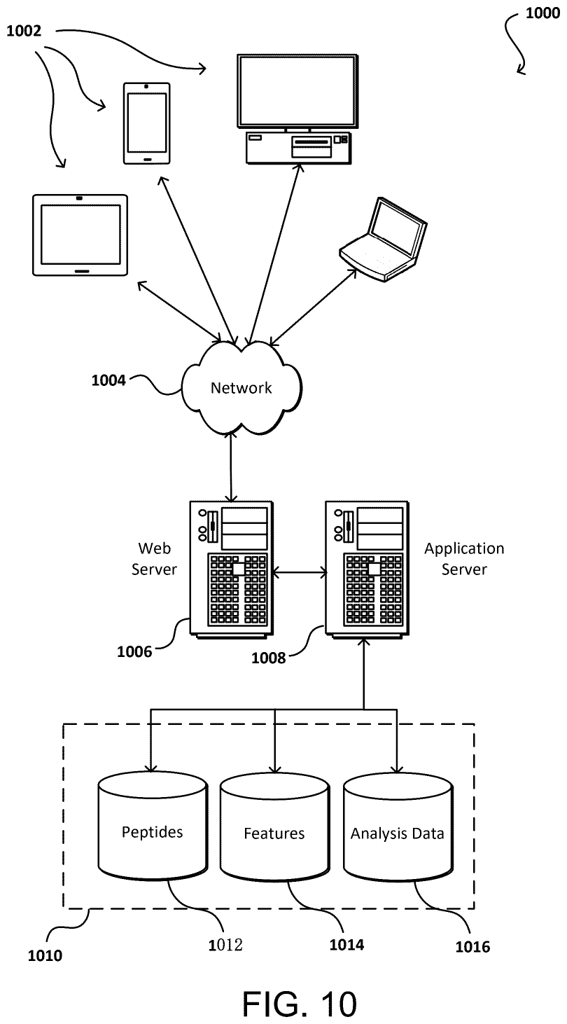

As discussed, different approaches can be implemented in various environments in accordance with the described embodiments. For example, FIG. 10 illustrates an example of an environment 1000 for implementing aspects in accordance with various embodiments. As will be appreciated, although a Web-based environment is used for purposes of explanation, different environments may be used, as appropriate, to implement various embodiments. The system includes an electronic client device 1002, which can include any appropriate device operable to send and receive requests, messages or information over an appropriate network 1004 and convey information back to a user of the device. Examples of such client devices include personal computers, cell phones, handheld messaging devices, laptop computers, set-top boxes, personal data assistants, electronic book readers and the like. The network can include any appropriate network, including an intranet, the Internet, a cellular network, a local area network or any other such network or combination thereof. Components used for such a system can depend at least in part upon the type of network and/or environment selected. Protocols and components for communicating via such a network are well known and will not be discussed herein in detail. Communication over the network can be enabled via wired or wireless connections and combinations thereof. In this example, the network includes the Internet, as the environment includes a Web server 1006 for receiving requests and serving content in response thereto, although for other networks, an alternative device serving a similar purpose could be used, as would be apparent to one of ordinary skill in the art.

The illustrative environment includes at least one application server 1008 and a data store 1010. It should be understood that there can be several application servers, layers or other elements, processes or components, which may be chained or otherwise configured, which can interact to perform tasks such as obtaining data from an appropriate data store. As used herein, the term “data store” refers to any device or combination of devices capable of storing, accessing and retrieving data, which may include any combination and number of data servers, databases, data storage devices and data storage media, in any standard, distributed or clustered environment. The application server 1008 can include any appropriate hardware and software for integrating with the data store 1010 as needed to execute aspects of one or more applications for the client device and handling a majority of the data access and business logic for an application. The application server provides access control services in cooperation with the data store and is able to generate content such as text, graphics, audio and/or video to be transferred to the user, which may be served to the user by the Web server 1006 in the form of HTML, XML or another appropriate structured language in this example. The handling of all requests and responses, as well as the delivery of content between the client device 1002 and the application server 1008, can be handled by the Web server 1006. It should be understood that the Web and application servers are not required and are merely example components, as structured code discussed herein can be executed on any appropriate device or host machine as discussed elsewhere herein.

The data store 1010 can include several separate data tables, databases or other data storage mechanisms and media for storing data relating to a particular aspect. For example, the data store illustrated includes mechanisms for storing peptides 1012 or peptide representations and analysis data 1016, which can be used to serve content for the production side. The data store is also shown to include a mechanism for storing feature data for the peptides 1014. It should be understood that there can be many other aspects that may need to be stored in the data store, such as manufacturing data or other training data, which can be stored in any of the above listed mechanisms as appropriate or in additional mechanisms in the data store 1010. The data store 1010 is operable, through logic associated therewith, to receive instructions from the application server 1008 and obtain, update or otherwise process data in response thereto. In one example, a user might submit a request for peptide synthesis. In this case, the data store might be used to access the peptide information, feature data, and analysis data to obtain information as to whether the peptide is synthesizable. The information can then be returned to the user, such as in a results listing on a Web page that the user is able to view via a browser on the user device 1002.

Each server typically will include an operating system that provides executable program instructions for the general administration and operation of that server and typically will include computer-readable medium storing instructions that, when executed by a processor of the server, allow the server to perform its intended functions. Suitable implementations for the operating system and general functionality of the servers are known or commercially available and are readily implemented by persons having ordinary skill in the art, particularly in light of the disclosure herein.

The environment in one embodiment is a distributed computing environment utilizing several computer systems and components that are interconnected via communication links, using one or more computer networks or direct connections. However, it will be appreciated by those of ordinary skill in the art that such a system could operate equally well in a system having fewer or a greater number of components than are illustrated in FIG. 10. Thus, the depiction of the system 1000 in FIG. 10 should be taken as being illustrative in nature and not limiting to the scope of the disclosure.

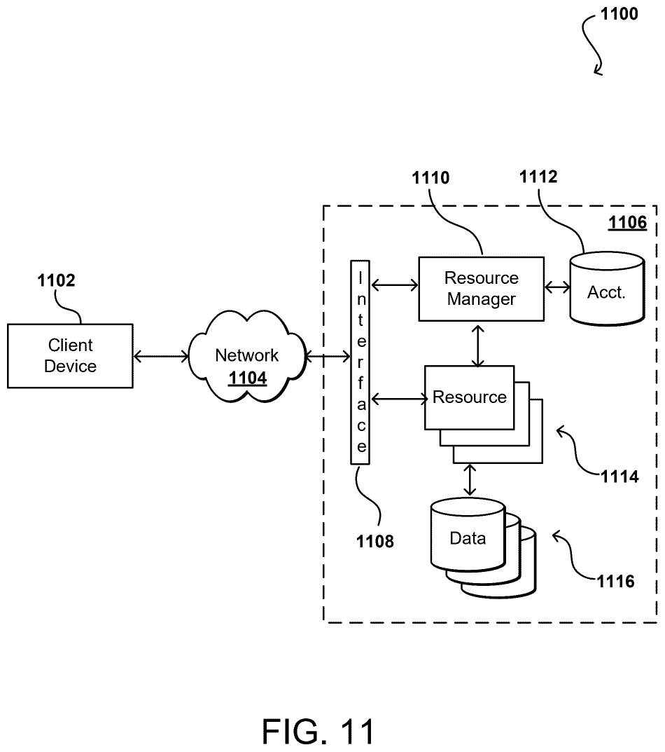

FIG. 11 illustrates an example environment 1100 in which aspects of the various embodiments can be implemented. In this example a user is able to utilize a client device 1102 to submit requests across at least one network 1104 to a multi-tenant resource provider environment 1106. The client device can include any appropriate electronic device operable to send and receive requests, messages, or other such information over an appropriate network and convey information back to a user of the device. Examples of such client devices include personal computers, tablet computers, smart phones, notebook computers, and the like. The at least one network 1104 can include any appropriate network, including an intranet, the Internet, a cellular network, a local area network (LAN), or any other such network or combination, and communication over the network can be enabled via wired and/or wireless connections. The resource provider environment 1106 can include any appropriate components for receiving requests and returning information or performing actions in response to those requests. As an example, the provider environment might include Web servers and/or application servers for receiving and processing requests, then returning data, Web pages, video, audio, or other such content or information in response to the request.

In various embodiments, the provider environment may include various types of resources that can be utilized by multiple users for a variety of different purposes. As used herein, computing and other electronic resources utilized in a network environment can be referred to as “network resources.” These can include, for example, servers, databases, load balancers, routers, and the like, which can perform tasks such as to receive, transmit, and/or process data and/or executable instructions. In at least some embodiments, all or a portion of a given resource or set of resources might be allocated to a particular user or allocated for a particular task, for at least a determined period of time. The sharing of these multi-tenant resources from a provider environment is often referred to as resource sharing, Web services, or “cloud computing,” among other such terms and depending upon the specific environment and/or implementation. In this example the provider environment includes a plurality of resources 1114 of one or more types. These types can include, for example, application servers operable to process instructions provided by a user or database servers operable to process data stored in one or more data stores 1116 in response to a user request. As known for such purposes, the user can also reserve at least a portion of the data storage in a given data store. Methods for enabling a user to reserve various resources and resource instances are well known in the art, such that detailed description of the entire process, and explanation of all possible components, will not be discussed in detail herein.

In at least some embodiments, a user wanting to utilize a portion of the resources 1114 can submit a request that is received to an interface layer 1108 of the provider environment 1106. The interface layer can include application programming interfaces (APIs) or other exposed interfaces enabling a user to submit requests to the provider environment. The interface layer 1108 in this example can also include other components as well, such as at least one Web server, routing components, load balancers, and the like. When a request to provision a resource is received to the interface layer 1108, information for the request can be directed to a resource manager 1110 or other such system, service, or component configured to manage user accounts and information, resource provisioning and usage, and other such aspects. A resource manager 1110 receiving the request can perform tasks such as to authenticate an identity of the user submitting the request, as well as to determine whether that user has an existing account with the resource provider, where the account data may be stored in at least one data store 1112 in the provider environment. A user can provide any of various types of credentials in order to authenticate an identity of the user to the provider. These credentials can include, for example, a username and password pair, biometric data, a digital signature, or other such information. The provider can validate this information against information stored for the user. If the user has an account with the appropriate permissions, status, etc., the resource manager can determine whether there are adequate resources available to suit the user's request, and if so can provision the resources or otherwise grant access to the corresponding portion of those resources for use by the user for an amount specified by the request. This amount can include, for example, capacity to process a single request or perform a single task, a specified period of time, or a recurring/renewable period, among other such values. If the user does not have a valid account with the provider, the user account does not enable access to the type of resources specified in the request, or another such reason is preventing the user from obtaining access to such resources, a communication can be sent to the user to enable the user to create or modify an account, or change the resources specified in the request, among other such options.

Once the user is authenticated, the account verified, and the resources allocated, the user can utilize the allocated resource(s) for the specified capacity, amount of data transfer, period of time, or other such value. In at least some embodiments, a user might provide a session token or other such credentials with subsequent requests in order to enable those requests to be processed on that user session. The user can receive a resource identifier, specific address, or other such information that can enable the client device 1102 to communicate with an allocated resource without having to communicate with the resource manager 1110, at least until such time as a relevant aspect of the user account changes, the user is no longer granted access to the resource, or another such aspect changes.

The resource manager 1110 (or another such system or service) in this example can also function as a virtual layer of hardware and software components that handles control functions in addition to management actions, as may include provisioning, scaling, replication, etc. The resource manager can utilize dedicated APIs in the interface layer 1108, where each API can be provided to receive requests for at least one specific action to be performed with respect to the data environment, such as to provision, scale, clone, or hibernate an instance. Upon receiving a request to one of the APIs, a Web services portion of the interface layer can parse or otherwise analyze the request to determine the steps or actions needed to act on or process the call. For example, a Web service call might be received that includes a request to create a data repository.

An interface layer 1108 in at least one embodiment includes a scalable set of user-facing servers that can provide the various APIs and return the appropriate responses based on the API specifications. The interface layer also can include at least one API service layer that in one embodiment consists of stateless, replicated servers which process the externally-facing user APIs. The interface layer can be responsible for Web service front end features such as authenticating users based on credentials, authorizing the user, throttling user requests to the API servers, validating user input, and marshalling or unmarshalling requests and responses. The API layer also can be responsible for reading and writing database configuration data to/from the administration data store, in response to the API calls. In many embodiments, the Web services layer and/or API service layer will be the only externally visible component, or the only component that is visible to, and accessible by, users of the control service. The servers of the Web services layer can be stateless and scaled horizontally as known in the art. API servers, as well as the persistent data store, can be spread across multiple data centers in a region, for example, such that the servers are resilient to single data center failures.

The various embodiments can be further implemented in a wide variety of operating environments, which in some cases can include one or more user computers or computing devices which can be used to operate any of a number of applications. User or client devices can include any of a number of general purpose personal computers, such as desktop or laptop computers running a standard operating system, as well as cellular, wireless and handheld devices running mobile software and capable of supporting a number of networking and messaging protocols. Such a system can also include a number of workstations running any of a variety of commercially-available operating systems and other known applications for purposes such as development and database management. These devices can also include other electronic devices, such as dummy terminals, thin-clients, gaming systems and other devices capable of communicating via a network.

Most embodiments utilize at least one network that would be familiar to those skilled in the art for supporting communications using any of a variety of commercially available protocols, such as TCP/IP, FTP, UPnP, NFS, and CIFS. The network can be, for example, a local area network, a wide-area network, a virtual private network, the Internet, an intranet, an extranet, a public switched telephone network, an infrared network, a wireless network and any combination thereof. In embodiments utilizing a Web server, the Web server can run any of a variety of server or mid-tier applications, including HTTP servers, FTP servers, CGI servers, data servers, Java servers and business application servers. The server(s) may also be capable of executing programs or scripts in response requests from user devices, such as by executing one or more Web applications that may be implemented as one or more scripts or programs written in any programming language, such as Java®, C, C# or C++ or any scripting language, such as Perl, Python or TCL, as well as combinations thereof. The server(s) may also include database servers, including without limitation those commercially available from Oracle®, Microsoft®, Sybase® and IBM®.

The environment can include a variety of data stores and other memory and storage media as discussed above. These can reside in a variety of locations, such as on a storage medium local to (and/or resident in) one or more of the computers or remote from any or all of the computers across the network. In a particular set of embodiments, the information may reside in a storage-area network (SAN) familiar to those skilled in the art. Similarly, any necessary files for performing the functions attributed to the computers, servers or other network devices may be stored locally and/or remotely, as appropriate. Where a system includes computerized devices, each such device can include hardware elements that may be electrically coupled via a bus, the elements including, for example, at least one central processing unit (CPU), at least one input device (e.g., a mouse, keyboard, controller, touch-sensitive display element or keypad) and at least one output device (e.g., a display device, printer or speaker). Such a system may also include one or more storage devices, such as disk drives, optical storage devices and solid-state storage devices such as random access memory (RAM) or read-only memory (ROM), as well as removable media devices, memory cards, flash cards, etc. Such devices can also include a computer-readable storage media reader, a communications device (e.g., a modem, a network card (wireless or wired), an infrared communication device) and working memory as described above. The computer-readable storage media reader can be connected with, or configured to receive, a computer-readable storage medium representing remote, local, fixed and/or removable storage devices as well as storage media for temporarily and/or more permanently containing, storing, transmitting and retrieving computer-readable information.

The system and various devices also typically will include a number of software applications, modules, services or other elements located within at least one working memory device, including an operating system and application programs such as a client application or Web browser. It should be appreciated that alternate embodiments may have numerous variations from that described above. For example, customized hardware might also be used and/or particular elements might be implemented in hardware, software (including portable software, such as applets) or both. Further, connection to other computing devices such as network input/output devices may be employed. Storage media and other non-transitory computer readable media for containing code, or portions of code, can include any appropriate media known or used in the art, such as but not limited to volatile and non-volatile, removable and non-removable media implemented in any method or technology for storage of information such as computer readable instructions, data structures, program modules or other data, including RAM, ROM, EEPROM, flash memory or other memory technology, CD-ROM, digital versatile disk (DVD) or other optical storage, magnetic cassettes, magnetic tape, magnetic disk storage or other magnetic storage devices or any other medium which can be used to store the desired information and which can be accessed by a system device. Based on the disclosure and teachings provided herein, a person of ordinary skill in the art will appreciate other ways and/or methods to implement the various embodiments.

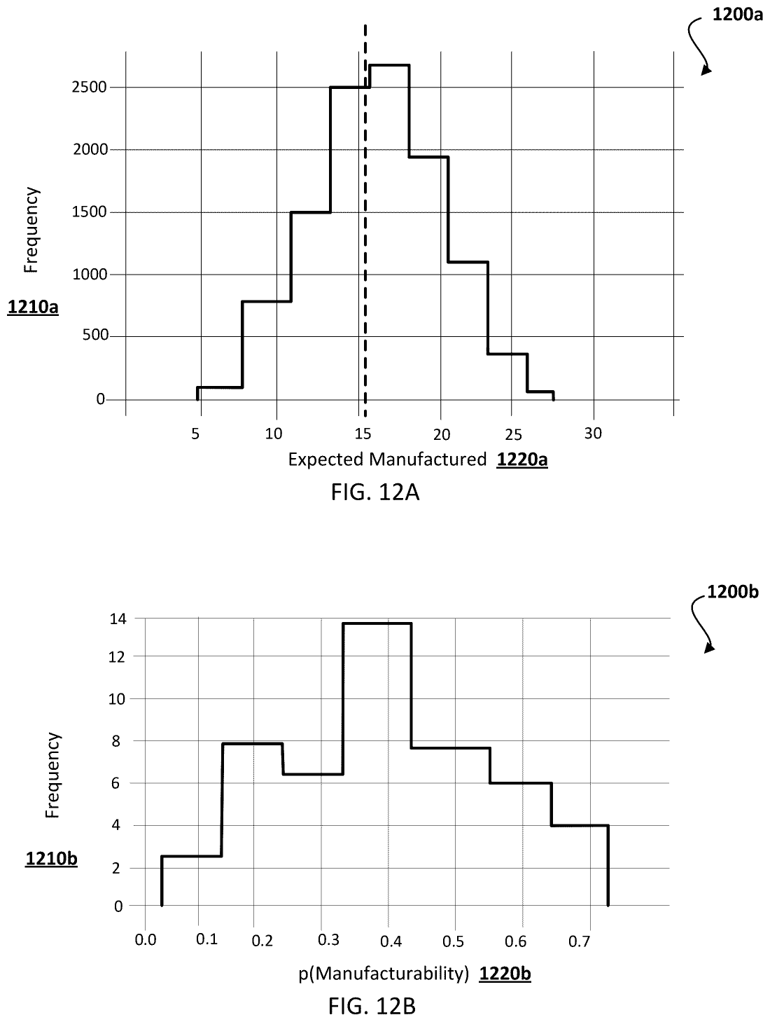

FIGS. 12A and 12B illustrate example histograms 1200a and 1200b for predicting a number of peptides to be successfully manufactured paired with the results from an attempt to manufacture those peptides. FIG. 12A is an example histogram illustrating the frequency at which certain expected numbers of a peptide's manufacturability occur within a dataset. The x-axis 1220a, labeled “Expected Manufactured,” can quantify the number of peptides that are expected to be successfully manufactured, while the y-axis 1210a indicates the binned frequency of these expectations across the dataset. The dashed vertical line represents the actual number of peptides manufactured, serving as a benchmark against the predicted values. FIG. 12A shows that the expected number of peptides to be successfully manufactured is 16.63, while the actual number of peptides that were successfully manufactured is 15. FIG. 12B is an example histogram illustrating the distribution of manufacturability probabilities (e.g., p(Manufacturability)). The x-axis 1220b represents the probability that a peptide can be manufactured (from 0 to 1), while the y-axis 1210b shows the binned frequency of peptides with these probabilities. FIG. 12B illustrates the confidence of the predictive model in terms of peptides' manufacturability. For example, for peptides with a high probability score, the model is confident that those peptides can be manufactured successfully. In one embodiment, the expected number of peptides anticipated to be manufactured is calculated by adding the pair-wise product between each frequency and probability. In other words, to calculate an overall expected number of manufacturable peptides for the entire dataset, the products of the probability of manufacturability for each peptide and the number of times that probability occurs can be summed. To calculate the expected number of peptides, each unique probability can be multiplied by the number of peptides assigned that probability, and then these products across all unique probability values can be summed.

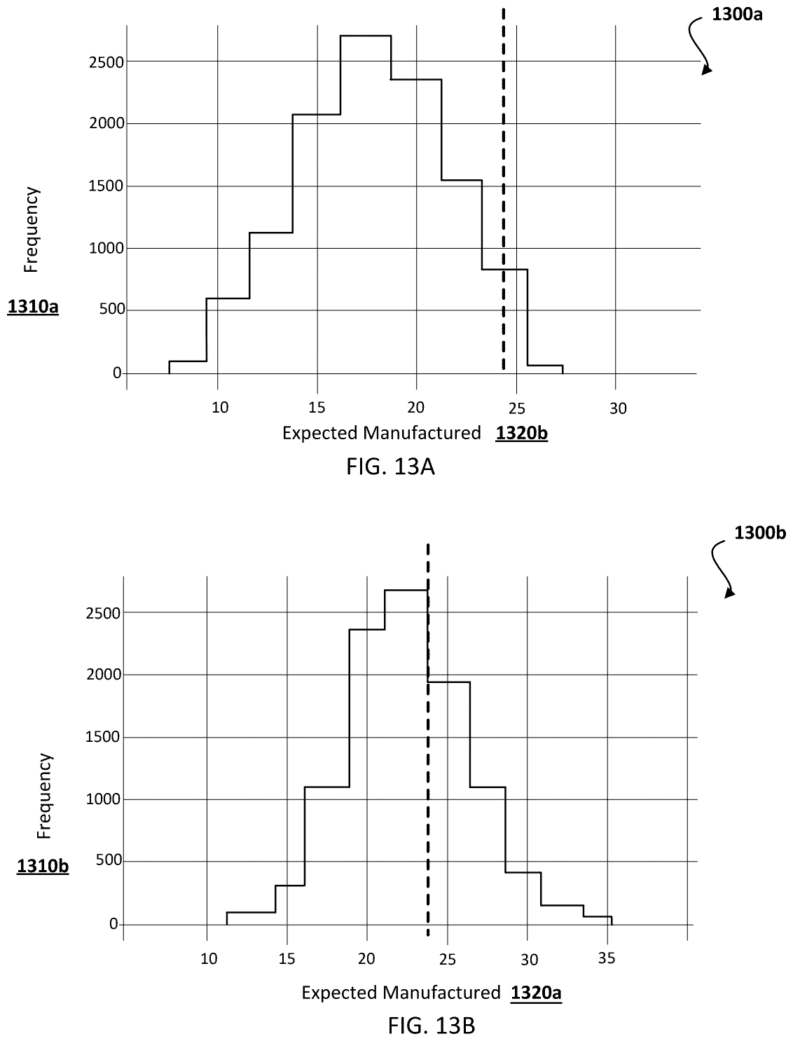

FIGS. 13A and 13B illustrate example histograms (1300a and 1300b) illustrating the expected numbers of peptides predicted to be manufactured and a comparison between two different methods using the same axes as 1200a. The vertical dashed lines indicate the actual number of peptides manufactured using the new method in both figures, where the actual number of peptides is 24. FIG. 13A illustrates a first method where peptides are first selected from a pool of peptides based solely on criteria other than manufacturability. The selected peptides can then be passed to the manufacturing simulation process, which results in an average of 17.59 peptides expected to be manufactured. FIG. 13B illustrates an improved method where the peptides are selected based on a combination of the manufacturability score and other criteria. The expected number manufactured using the second method is about 22.90 and the when peptides were selected by this method, 24 of them were manufactured.

The specification and drawings are, accordingly, to be regarded in an illustrative rather than a restrictive sense. It will, however, be evident that various modifications and changes may be made thereunto without departing from the broader spirit and scope of the invention as set forth in the claims.

1. A computer-implemented method, comprising:
receiving a request for information related to manufacturability of a peptide;
determining whether the peptide is predicted to be synthesizable;
determining whether the peptide is predicted to be soluble; and
determining, based at least in part on whether the peptide is predicted to be synthesizable and soluble, whether the peptide is manufacturable using a trained machine learning model.

2. The computer-implemented method of claim 1, further comprising:
determining whether the peptide is predicted to pass a quality control check; and
determining, based at least in part on whether the peptide is predicted to be synthesizable, soluble, and pass the quality control check, whether the peptide is manufacturable.

3. The computer-implemented method of claim 1, further comprising:
calculating a manufacturability score for the peptide, the manufacturability score indicative of a probability of successfully manufacturing the peptide; and
using the manufacturability score to determine whether the peptide is manufacturable.

4. The computer-implemented method of claim 1, wherein the machine learning model is trained, at least in part, on peptide data, peptide feature data, manufacturer specification and descriptor data, or manufacturability data.

5. The computer-implemented method of claim 1, wherein a result of whether the peptide was successfully manufactured is used to further train the machine learning model.

6. The computer-implemented method of claim 1, wherein the determining whether the peptide is predicted to be synthesizable and determining whether the peptide is predicted to be soluble are each performed using models trained specifically for each determination.

7. The computer-implemented method of claim 1, further comprising:
selecting the peptide for manufacture based on the determination whether the peptide is manufacturable.

8. A computing system, comprising:
a computing device processor; and
a memory device including instructions that, when executed by the computing device processor, enable the computing system to:
receive a request for information related to manufacturability of a peptide;
determine whether the peptide is predicted to be synthesizable;
determine whether the peptide is predicted to be soluble; and
determine, based at least in part on whether the peptide is synthesizable and soluble, whether the peptide is manufacturable using a trained machine learning model.

9. The computing system of claim 8, wherein the instructions that, when executed by the computing device processor, enable the computing system to further:
determine whether the peptide is predicted to pass a quality control check; and
determine, based at least in part on whether the peptide is predicted to be synthesizable, soluble, and pass the quality control check, whether the peptide is manufacturable.

10. The computing system of claim 8, wherein the instructions that, when executed by the computing device processor, enable the computing system to further:
calculate a manufacturability score for the peptide, the manufacturability score indicative of a probability of successfully manufacturing the peptide; and
use the manufacturability score to determine whether the peptide is manufacturable.

11. The computing system of claim 8, wherein the machine learning model is trained, at least in part, on peptide data, peptide feature data, manufacturer specification and descriptor data, or manufacturability data.

12. The computing system of claim 8, wherein a result of whether the peptide was successfully manufactured is used to further train the machine learning model.

13. The computing system of claim 8, wherein the determining whether the peptide is predicted to be synthesizable and determining whether the peptide is predicted to be soluble are each performed using models trained specifically for each determination.

14. The computing system of claim 8, wherein the determining whether the peptide is predicted to be synthesizable and the determining whether the peptide is predicted to be soluble is determined as part of using the trained machine learning model to determine whether the peptide is manufacturable.

15. The computing system of claim 8, wherein the instructions that, when executed by the computing device processor, enable the computing system to further:
determine a solubility score for the peptide;
determine a synthesizability score for the peptide; and
predict a likelihood of manufacturability for the peptide based, at least in part, on the solubility score or the synthesizability score.

16. A non-transitory computer-readable medium comprising instructions stored thereon, that when executed on a processor, perform the steps of:
receiving a request for information related to manufacturability of a peptide;
determining whether the peptide is predicted to be synthesizable;
determining whether the peptide is predicted to be soluble; and
determining, based at least in part on whether the peptide is predicted to be synthesizable and soluble, whether the peptide is manufacturable using a trained machine learning model.

17. The non-transitory computer-readable medium of claim 16, wherein the instructions that, when executed on the processor, additionally perform the steps of:
determining whether the peptide is predicted to pass a quality control check; and
determining, based at least in part on whether the peptide is predicted to be synthesizable, soluble, and pass the quality control check, whether the peptide is manufacturable.

18. The non-transitory computer-readable medium of claim 16, wherein the instructions that, when executed on the processor, additionally perform the steps of:
determining a solubility score for the peptide;
determine a synthesizability score for the peptide; and
predicting a likelihood of manufacturability for the peptide based, at least in part, on the solubility score or the synthesizability score.

19. The non-transitory computer-readable medium of claim 16, wherein the machine learning model is trained, at least in part, on peptide data, peptide feature data, manufacturer specification and descriptor data, or manufacturability data.

20. The non-transitory computer-readable medium of claim 16, wherein a result of whether the peptide was successfully manufactured is used to further train the machine learning model.
